(19) United States
(12) Patent Application Publication
(10) Pub. No.: US 2026/0215722 A1
(43) Pub. Date: Jul. 30, 2026

(54) SYSTEMS AND METHODS FOR ASSESSING BRAIN HEALTH

(71) Applicant: REDENLAB PTY. LTD., Melbourne, Victoria (AU)

(72) Inventors: Adam Phillip Reginald Vogel, Melbourne, Victoria (AU); Devon Leonard Borysiewicz, Melbourne, Victoria (AU)

(21) Appl. No.: 19/142,023

(22) PCT Filed: Dec. 22, 2023
(86) PCT No.: PCT/AU2023/051356

§ 371 Date: Jun. 20, 2025

(30) Foreign Application Priority Data
Dec. 22, 2022 (AU) 2022903954

(51) Int. Cl. A61B 5/00

(52) U.S. Cl. CPC A61B 5/4064; A61B 5/4803; A61B 5/7264

(57) ABSTRACT

Embodiments generally relate to systems and computer implemented methods for assessing brain health. One method may comprise: receiving an audio recording of sounds made by a participant; determining, from the audio recording, a textual representation of at least one sound made by the participant; providing the audio recording to an acoustic analysis model; receiving from the acoustic analysis model, a speech quality dataset comprising one or more speech quality metric(s); providing the textual representation to a text analysis model; receiving from the text analysis model, a speech content dataset comprising one or more speech content metric(s); and determining, from the speech quality dataset and the speech content dataset a speech score associated with the participant; wherein the speech score is indicative of a brain health of the participant.

### TECHNICAL FIELD

Described embodiments relate to computing systems and computer implemented methods for assessing brain health. In some embodiments, the computing systems and computer implemented methods relate to speech and language analysis for assessing brain health.

### BACKGROUND

Speech analysis for the determination or assessment of altered brain health is a specialised area of medicine and psychology requiring extensive training to be performed by a practicing physician or clinician. Prior solutions of one-on-one sessions with a trained expert are time intensive, expensive and/or inaccessible to many individuals afflicted with brain health problems that require either diagnosis or ongoing treatment and management. Accurate identification and assessment of altered brain health resulting from tiredness or stress is challenging. Objective measurement of these performance markers is not readily available or easy to operationalise.

It is desired to address or ameliorate some of the disadvantages associated with such prior methods and systems, or at least to provide a useful alternative thereto.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

The present disclosures are directed to a computer implemented method comprising: receiving an audio recording of sounds made by a participant; determining, from the audio recording, a textual representation of at least one sound made by the participant; providing the audio recording to an acoustic analysis model; receiving from the acoustic analysis model, a speech quality dataset comprising one or more speech quality metric(s); providing the textual representation to a text analysis model; receiving from the text analysis model, a speech content dataset comprising one or more speech content metric(s); and determining, from the speech quality dataset and the speech content dataset a speech score associated with the participant; wherein the speech score is indicative of a brain health of the participant.

In some embodiments, the speech score may comprise at least a naturalness score. The naturalness score may include at least one measure of: fundamental frequency variability, intensity variability, formant transitions, speech rate and rhythm, phonation measures, spectral measures, temporal measures, articulation, coarticulation, resonance and prosody. In some embodiments, the speech score may comprise at least an intelligibility score.

In some embodiments, the speech content dataset comprises at least a discourse complexity metric. The discourse complexity metric may include at least one measure of: lexical diversity, syntactic complexity, referential cohesion, thematic development, argumentative structure, implicit and explicit information comparison, interactivity, intertextuality, modality and modulation and pragmatic factors.

In some embodiments, the speech score comprises one or more: communication effectiveness score, dysarthria score, disease severity score, social communication score, voice quality score, intelligibility score and/or naturalness score.

In some embodiments, the speech quality dataset comprises one or more: timing metric, articulation metric, resonance metric, prosody metric and/or voice quality metric.

In some embodiments, the speech content dataset comprises one or more: semantic complexity metrics, idea density metrics, verbal fluency metrics, lexical diversity metrics, informational content metrics, discourse structure metrics, and/or grammatical complexity metrics.

In some embodiments, the method further comprises comparing the speech score associated with the participant, to a control speech score to determine the brain health of the participant. In some embodiments, the control speech score is experimentally determined or statistically determined.

In some embodiments, the sounds made by a participant are spoken words and/or sounds; and wherein the spoken words and/or sounds are in response to a speech task provided to the participant and/or the spoken words and/or sounds were recorded over a period of continuous observation of the participant without being prompted.

In some embodiments, the disclosed methods may comprise performing data quality assurance.

In some embodiments, the data quality assurance comprises one or more of: (a) determining if a voice is present; (b) determining a recording duration; (c) determining the recording duration is within a predetermined recording limit; (d) remove aberrant noise; (e) remove silence; (f) determine the number of recorded speakers (g) separate speakers.

In some embodiments, if the audio recording does not pass the data quality assurance step, sending a notification to a mobile computing device.

In some embodiments, the determination of the speech content score and the speech quality score occur in parallel.

In some embodiments, the disclosed methods further comprise receiving one or more narrowing parameters. The one or more narrowing parameters comprising: one or more brain health conditions; one or more brain health attributes; one or more brain health metrics; one or more neurological disorders; and/or participant medical history, in some embodiments.

In some embodiments, responsive to receiving one or more narrowing parameters, adjusting the acoustic analysis model and/or the text analysis model such that the speech quality dataset and/or the speech content dataset are associated with the one or more narrowing parameters.

In some embodiments, one or more of the textual representation, the speech quality dataset, the speech content dataset and the speech score are determined by one or more machine learning model(s). In some embodiments, at least one of the one or more machine learning model(s) is a neural network.

In some embodiments, the disclosed methods further comprise the step of converting the textual representation into a word embedding.

In some embodiments, the method further comprises the step of: before the audio recording is provided to the acoustic analysis model, converting the audio recording into one or more of a series of filter-bank spectra and/or one or more sonic spectrogram.

In some embodiments, the textual representation is determined using natural language processing.

In some embodiments, the method further comprises the step of: determining that no textual representation can be determined; and wherein upon determining that no textual representation can be determined, skipping the step of determining the textual representation. In some embodiments, determining that no textual representation can be determined comprises: determining that the audio recording does not contain at least a single morpheme, phoneme or sound that can be represented textually.

In some embodiments, subsequent to determining that the audio recording does not contain at least a single morpheme or sound that can be represented textually; determining an indication that the audio recording does not contain at least a single morpheme, phoneme or sound that can be represented textually; and wherein the indication is used as an input for determining the speech score.

The present disclosures are also directed to a non-transitory machine-readable medium storing instructions which, when executed by one or more processors, cause a computing device to perform the method of any of the present disclosures.

The present disclosures are also directed to a system for assessing brain health comprising: a speech analysis module configured to determine a speech quality dataset from an audio recording; a speech to text module configured to determine a textual representation of the audio recording; a speech content module configured to determine a speech content dataset from the audio recording; a brain health determination module configured to determine, using the speech quality dataset and the speech content dataset, a speech score; wherein the speech score is indicative of the brain health of the one or more participants recorded on the audio recording.

In some embodiments, the system further comprises a screen, configured to present instructions to the one or more participants.

In some embodiments, the system further comprises an audio recording device, configured to record the one or more participants.

In some embodiments, the speech analysis module comprises one or more machine learning models and/or information sieves. In some embodiments, the speech to text module comprises a machine learning model. In some embodiments, the speech content module comprises a machine learning model. In some embodiments, the machine learning models are recurrent neural networks.

The present disclosures are also directed to a computer implemented method comprising: receiving an audio recording of sounds made by a participant; providing the audio recording to an acoustic analysis model; receiving from the acoustic analysis model, a speech quality dataset comprising one or more speech quality metric(s); determining, from the speech quality dataset a speech score associated with the participant; wherein the speech score is indicative of a brain health of the participant.

### BRIEF DESCRIPTION OF DRAWINGS

FIG. 1 is a schematic diagram of a broad overview of a method of assessing brain heath according to some embodiments.

FIG. 2 is a block diagram of a system for assessing brain health, according to some embodiments;

FIG. 3 is a block diagram of an alternative system for assessing brain health, according to some embodiments;

FIG. 4 is a process flow diagram of a method of assessing brain health, according to some embodiments;

FIGS. 5A, B and C are example screenshots of a user interface for assessing brain health, according to some embodiments; and

FIG. 6 is a process flow diagram of a method of conducting brain health assessment, according to some embodiments.

### DESCRIPTION OF EMBODIMENTS

Described embodiments relate to computing systems and computer implemented methods for assessing brain health. Some embodiments relate to the use of audio and/or text analysis to determine a speaker's neurological status and/or condition. Some embodiments may comprise one or more machine learning models to conduct the speech and/or text analysis.

In relation to the present disclosures, brain health may include or otherwise be defined as the presence and/or state of a participant's brain integrity and mental and cognitive function at a given age in the presence or absence of overt brain diseases that affect normal brain function. Brain health may be and/or be measured as the ability to perform all, most, some or none the mental processes of cognition, including the ability to learn and judge, use language, and remember. In some embodiments, brain health may be a statistically defined, i.e. defined by studying a population of patients. In some embodiments, brain health may be a relative measure of a single patient, compared to previous measurements. Brain health may or may not be associated with and/or related to the presence of a diagnosed or undiagnosed neurological condition.

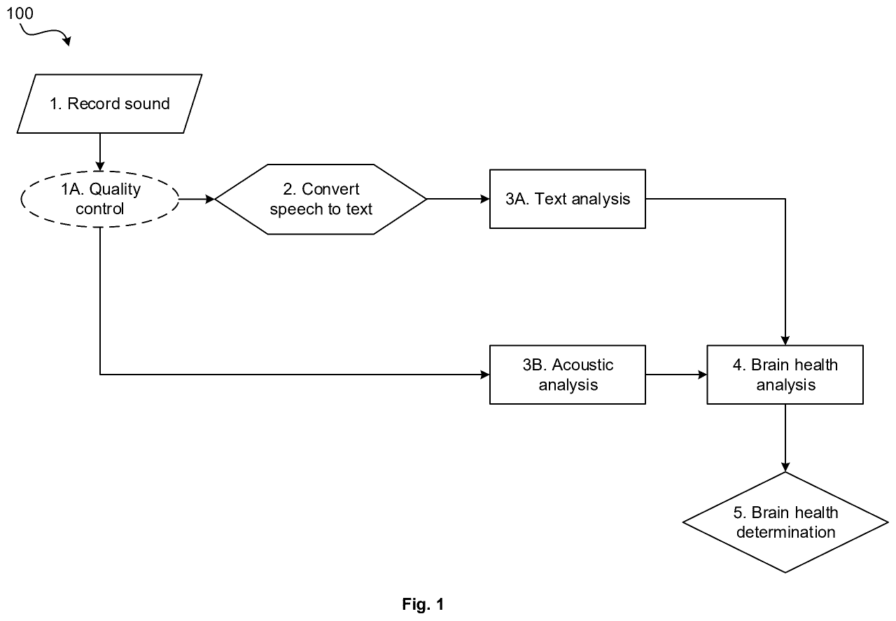

FIG. 1 is a schematic diagram, depicting a broad overview of a method 100 performed by the system of the present disclosures.

According to the described embodiments, at step 1 as shown in FIG. 1, the sounds a participant creates are recorded. The sounds the participant creates may be created by the participant in response to some form of stimulus. In some embodiments, the participant may be presented one or more speech tasks and/or recording tasks that require them to speak a certain phrase, hold a conversation, make a certain sound, or record sounds for a determined or undetermined amount of time. The participant may be presented the speech task via a mobile computing device such as smart phone, tablet, laptop, or computer kiosk. In some embodiments, the participant may not be presented with a particular speech task and instead may be presented with a recording task. A recording task may comprise continuously recording for an extended period of time, so as to record any sounds and/or utterances the participant may make naturally and/or unprompted. Unprompted sounds and/or utterances may comprise coughing, breathing, vomiting, wheezing and/or non-descript muttering.

At step 1A, the recorded sounds may undergo one or more processes of quality control to improve and/or remove sound recordings that may not be sufficient for brain health determination.

At step 2, the recorded sounds are converted to text. In some embodiments, any speech, spoken words and/or non-lexical utterances/conversational sounds such as ‘uh-huh, ‘hmm’, ‘erm’ and/or throat clearing, present in the recorded sound may be converted into text/a textual representation. The textual representation created at step 2 and the recorded sound as created at step 1 may be fed to a text analysis module and an acoustic analysis module respectively.

At step 3A, the textual representation created at step 2 undergoes text analysis by the text analysis module. At step 3B, the recorded sound as created at step 1 undergoes acoustic analysis by the acoustic analysis module. The text analysis module and acoustic analysis module may avail of one or more machine learning models to output a speech content dataset and speech quality dataset, respectively. The datasets may be descriptive of various qualities of the recorded sound.

At step 4, the speech content dataset and speech quality dataset are used by another machine learning model to perform brain health analysis.

At step 5, the result of the brain health analysis is a determination of the brain health of the participant. The participant's brain health determination may be indicative of whether the participant has a neurological disorder or be indicative of the progression of an existing neurological disorder. The brain health determination may also indicate a level of the participant's brain health. This level of the participant's brain health may be on a scale, from healthy to abnormal or unhealthy, or may be a binary determination associated with a brain health threshold, for example.

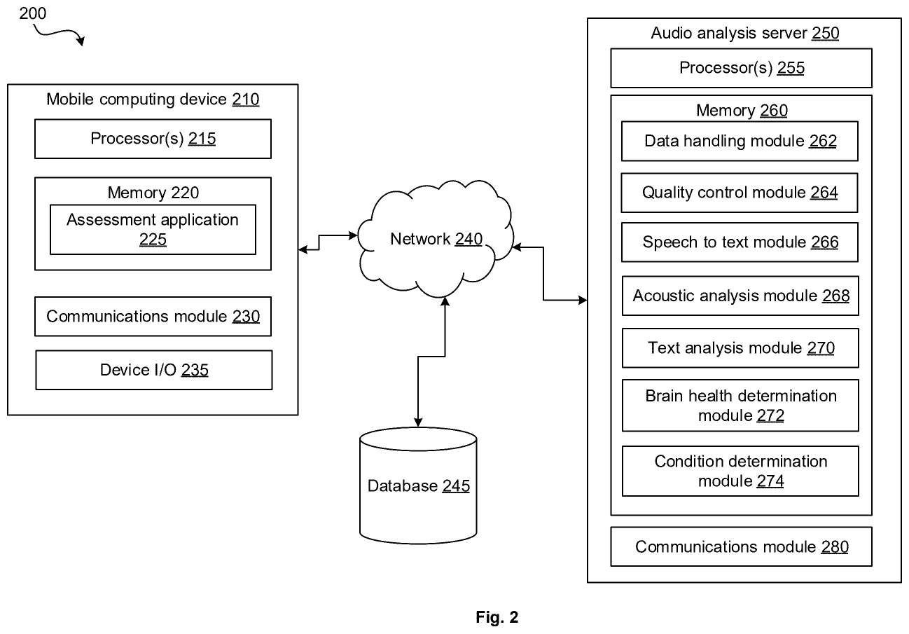

FIG. 2 is a block diagram of a system 200 for assessing brain health, according to some embodiments. System 200 may comprise mobile computing device 210, database 245 and audio analysis server 250, in communication via a network 240. The network 240 may include, for example, at least a portion of one or more networks having one or more nodes that transmit, receive, forward, generate, buffer, store, route, switch, process, or a combination thereof, etc. one or more messages, packets, signals, some combination thereof, or so forth. The network 240 may include, for example, one or more of: a wireless network, a wired network, an internet, an intranet, a public network, a packet-switched network, a circuit-switched network, an ad hoc network, an infrastructure network, a public-switched telephone network (PSTN), a cable network, a cellular network, a satellite network, a fibre-optic network, some combination thereof, or so forth.

The mobile computing device 210 may be a mobile or handheld computing device such as a smartphone or tablet, a laptop, smartwatch, passive room based microphone, or a PC, and may, in some embodiments, comprise multiple computing devices. Mobile computing device 210 may comprise one or more processor(s) 215, and memory 220 storing instructions (e.g. program code) executable by the processor(s) 215. The processor(s) 215 may comprise one or more microprocessors, central processing units (CPUs), application specific instruction set processors (ASIPs), application specific integrated circuits (ASICs) or other processors capable of reading and executing instruction code.

In some embodiments, the mobile computing device 210 and the audio analysis server 250 may have a client-server architecture. The mobile computing device 210 may be a thin client, tasked with presentation logic and input output management, to present the speech tasks and/or recording tasks and collect the audio recordings, with all or most logic operations and data storage operations related to processing the audio recordings being handled by the audio analysis server 250. For example, the thin client may be configured to receive data packets from the audio analysis server 250 via network 240, that when received by communications module 230 and executed by processor(s) 215 may cause the thin client to do one or more of: display text, images and/or interactive elements on a screen, or screens of the mobile computing device; and/or accept input from one or more users in the form of a button press, screen interaction, video recordings and/or audio recordings.

In some embodiments, the mobile computing device 210 may be a thick client, capable of collecting, storing and/or pre-processing the audio recordings before they are communicated to the audio analysis server 250. For example, the thick client may comprise one or more modules of executable program code that when executed by the processor(s) 215 may cause the thick client to pre-process the audio recording to conduct quality control on the audio recordings, such as described below. The thick client may also be configured to prompt the user(s)/participants to re-record speech tasks or recommence extended recording if the audio recording fails one or more of the quality control metrics.

Memory 220 may comprise one or more volatile or non-volatile memory types. For example, memory 220 may comprise one or more of random access memory (RAM), read-only memory (ROM), electrically erasable programmable read-only memory (EEPROM) or flash memory. Memory 220 is configured to store program code accessible by the processor(s) 215. The program code comprises executable program code modules. In other words, memory 220 is configured to store executable code modules configured to be executable by the processor(s) 215. The executable code modules, when executed by the processor(s) 215, may cause processor(s) 215 to perform various methods as described in further detail below. Memory 220 may comprise an assessment application 225.

Assessment application 225 may be configured to present and manage an assessment process. The assessment application 225 may be a piece or pieces of software that may be downloadable from a website, application store and/or external data store. In some embodiments, the assessment application 220 may be preinstalled.

Mobile computing device 210 may comprise communications module 230. The communications module 230 facilitates communications with components of the system 200 across the network 240, such as: audio analysis server 250 and/or database 245.

Mobile computing device 210 may also comprise device I/O 235, configured to record, or accept as an input, sounds such as spoken words, for example. In some embodiments, mobile computing device may be configured to record and store any input sounds, and cause these to be communicated to audio analysis server 250, via network 240, for example. In some embodiments, mobile computing device 210 may be configured to stream any input sounds directly to audio analysis server 250. In some embodiments, mobile computing device may be configured to communicate audio inputs to database 245, for storage and eventual processing by audio analysis server 250. Device I/O 235 may comprise one or more further user input or user output peripherals, such as one or more of a display screen, touch screen display, camera, accelerometer, mouse, keyboard, or joystick, for example.

The network 240 may comprise a combination of network interface hardware and network interface software suitable for establishing, maintaining and facilitating communication over a relevant communication channel. The network 240 may include, for example, at least a portion of one or more networks having one or more nodes that transmit, receive, forward, generate, buffer, store, route, switch, process, or a combination thereof, etc. one or more messages, packets, signals, some combination thereof, or so forth. The network 240 may include, for example, one or more of: a wireless network, a wired network, an internet, an intranet, a public network, a packet-switched network, a circuit-switched network, an ad hoc network, an infrastructure network, a public-switched telephone network (PSTN), a cable network, a cellular network, a satellite network, a fibre-optic network, some combination thereof, or so forth.

The database 245, which may form part of or be local to the system 200, or may be remote from and accessible to the system 200, for example, via the network 240. The database 245 may be configured to store data associated with the system 200. The database 245 may be a centralised database. The database 245 may be a mutable data structure. The database 245 may be a shared data structure. The database 245 may be a data structure supported by database systems such as one or more of PostgreSQL, MongoDB, and/or ElasticSearch. The database 245 may be configured to store a current state of information or current values associated with various attributes (e.g., “current knowledge”).

In some embodiments, the database 245 may be an SQL database comprising tables with a line entry for each user of the system 200. For example, the line item may comprise entries for a participant's name, a participant's password, participant's brain health determination, participant's speech scores and/or other entries relating to a user's brain health.

Audio analysis sever 250 may comprise one or more processors 255 and memory 260 storing instructions (e.g. program code) which when executed by the processor(s) 255 causes the audio analysis server 250 to function according to the described methods. In some embodiments, the audio analysis server 250 may operate in conjunction with or support one or more mobile computing devices 210, to enable the brain health determination process and in some embodiments, provide a determination the user once the inputs have been suitably analysed.

The processor(s) 255 may comprise one or more microprocessors, central processing units (CPUs), application specific instruction set processors (ASIPs), application specific integrated circuits (ASICs) or other processors capable of reading and executing instruction code.

Memory 260 may comprise one or more volatile or non-volatile memory types. For example, memory 260 may comprise one or more of random access memory (RAM), read-only memory (ROM), electrically erasable programmable read-only memory (EEPROM) or flash memory. Memory 260 is configured to store program code accessible by the processor(s) 255. The program code comprises executable program code modules. In other words, memory 260 is configured to store executable code modules configured to be executable by the processor(s) 255. The executable code modules, when executed by the processor(s) 255 cause the audio analysis server 250 to perform certain functionality, as described in more detail below. For example, memory 260 may comprise data handling module 262, quality control module 264, speech to text module 266, acoustic analysis module 268, text analysis module 270, brain health determination module 272, and/or condition determination module 274.

Audio analysis server 250 may also comprise communications module 280. The communications module 280 facilitates communications with components of the system 200 across the network 240. The communications module 280 may comprise a combination of network interface hardware and network interface software suitable for establishing, maintaining and facilitating communication over a relevant communication channel.

Data handling module 262, is configured to receive and/or request data from other components in system 200, such as mobile computing device 210 or database 245. In some embodiments data handling module may be configured to receive and/or request data from devices outside of the system 200, such as an external database belonging to a medical records service provider, or an external subject testing system, for example.

Data handling module 262 may receive data in the form of audio recordings. In some embodiments, the audio records are of one or more persons speaking or producing biological sounds. The one or more persons speaking may be a subject of medical testing, a general practitioner, a nurse, neurologist, a family member, a dedicated carer or any other type of person who is capable of administrating and/or taking part in the speech task, recording task and/or brain health determination process.

In some embodiments, data handling module may be configured to receive audio recordings of one or more persons speaking, via network 240. The audio recordings may be a .WAV file, a .PCM file, an .AIFF file, an .MP3 file, or a .WMA file, in some embodiments. Data handling module may, in some embodiments, be configured to temporarily store received audio recordings for subsequent communication to other modules of memory 260.

The data handling module 262 in conjunction with the communications module 280 may be configured to monitor the receipt of individual data packets received over network 240. In some embodiments, the audio recording may be transmitted using a lossless data transmission protocol, such as transmission control protocol (TCP), to ensure audio file integrity. Data handling module 262 may request certain packets be resent if a specific TCP packet or packets are not received. Mobile computing device 210, database 245 and/or other external device may be configured to resend lost packets upon receiving an indication from the data handling module 262, that a packet has not been received.

In some embodiments, where network connectivity and/or bandwidth may be limited, or when data transfer speed is preferred over data integrity, the audio recordings may be transmitted using a lossy data transmission protocol, such as user datagram protocol (UDP).

Quality control module 264, may be configured to receive one or more audio recordings from data handling module 262. The quality control module 264 is configured to determine if the audio recordings are suitable for use in determining/assessing brain health. The quality control process may comprise determining if the audio recording is suitable for use in determining brain health by conducting one or more quality tests on the audio recording. The quality tests may include, but may not be limited to: checking if the sound file is empty; detecting whether other speakers additional to the primary speaker have been recorded; checking stimuli produced aligns with target stimuli according to one or more selected task and the associated minimum requirements; and/or checking a signal to noise ratio of the recording.

In some embodiments, the one or more quality tests may each have its own pass threshold that is indicative of an acceptable level of the quality that test is indicative of. Some quality tests may be binary pass/fail tests, such as whether the audio file is empty or not. For some quality tests, the pass threshold may be a proportion of the recording that contains a certain unwanted quality, for example, an audio recording may pass a quality test if the signal to noise ratio is less than 50%, for example. Other quality tests may have pass thresholds related to the intensity of a particular feature of the audio recording, for example, if a particular quality test is assessing the presence of two or more speakers, if the additional speaker's voice does not reach a minimum decibel rating, the audio recording may fail that particular quality test.

In some embodiments, the quality control module 264 may also conduct one or more quality improvement processes, including but not limited to: filtering out background noise such as clicks, static and/or audio artefacts; where voice intensity is concerned, equalising speech intensity to assist analysis of things such as syllable emphasis; and/or clipping periods of extended silence from the audio file, such as large pauses in speech and silence at the beginning and end of the recording.

In some embodiments, if the quality control module 264 determines that the audio recording is not of sufficient quality, for example by the audio recording failing to pass (e.g. not meeting the pass threshold) of a certain number of quality checks, the quality control module may be configured to mark the particular audio recording as not suitable. In some embodiments, when an audio recording is determined to not be suitable, the quality control module 264 may communicate a notification/indication to the mobile computing device 210, indicating that a particular audio recording is of unacceptable quality. In some embodiments, the notification/indication may also include a prompt to re-record the particular audio recording by redoing the associated speech task, or recommencing/restarting the extended recording of audio.

The speech to text module 266 may be configured to receive as an input the audio recording of sounds made by a participant, and output text that is a representation of the sounds made by the participant. The representation of the sounds made by the participant may be a direct transcription of the spoken words into written words. In some embodiments, the written words may not be a direct transcription, but a representation of the spoken words. The representation may have repeated words, stutters, and/or large pauses removed. Where spoken words are unintelligible, the speech to text module 266 may do one or more of: not include the unintelligible spoken words; replace the unintelligible words with an indication that the written words cannot be determined; include a best guess of the spoken word; and/or a list of possible words based on the qualities of the unintelligible spoken words and/or the context of the sentence the unintelligible words belong to, for example.

Acoustic analysis module 268, is configured to determine, from the audio recording a speech quality dataset, comprising one or more speech quality metrics. The one or more speech quality metrics may be indicative of the quality of the way in which the participant is speaking. The quality of a participant's speech may be unrelated to the content of their speech, and may include metrics such as, the speed of their speech, pauses between words, stuttering, pitch and/or articulation, for example.

Text analysis module 270, is configured to determine, from the textual representation of the sounds made by the participant, a speech content dataset, comprising one or more speech content metrics. The speech content metrics may be indicative of the content of the participant's speech. The content of a participant's speech may be unrelated to the quality of the participant's speech, and may include metrics such as lexical complexity, syllabic complexity, correct/incorrect pronunciations, repetition, informational efficiency and/or grammatical complexity, semantic complexity metrics, idea density metrics, verbal fluency metrics, lexical diversity metrics, informational content metrics, and/or discourse structure metrics, for example. One or more of these metrics may be used to form discourse complexity metrics. In some embodiments, the speech content dataset may include discourse complexity metrics.

Discourse complexity may also be referred to as conversational intricacy, discursive depth, dialogical complexity, talk sophistication, verbal complexity, narrative intricacy, rhetorical depth, linguistic complexity in discourse, discussion intricateness, communicative sophistication, and/or communicative effectiveness. The discourse complexity metric refers to the multidimensionality and intricate nature of communicative texts or spoken language. In some embodiments, discourse complexity metrics may include structural, conceptual and/or relational components of speech that interact within a communicative event.

In some embodiments, discourse complexity metrics may be comprised of one or more aspects that contribute to the metric. For example, lexical diversity metrics may be considered a discourse complexity metric. In some embodiments, discourse complexity metrics may include a plurality of different metrics which are used in combination to provide a discourse complexity metric. The plurality of metrics may each be weighted differently to contribute to the discourse complexity metric. In another embodiment, the plurality of metrics, or a subset of the plurality of metrics, may each be weighted substantially equally to contribute to the discourse complexity metric. For example, a weighting function may be used to appropriately weight each individual metric in the discourse complexity metric based on the circumstances of the assessment.

In some embodiments, the discourse complexity metric may include lexical diversity which takes into account the range and variety of words used. In some embodiments, the discourse complexity metric may include syntactic complexity in which the use of diverse and advanced sentence structures is considered. In some embodiments, the discourse complexity metric may include referential cohesion which is the consistency and clarity with which subjects, objects and ideas are connected and referenced throughout the discourse. In some embodiments, the discourse complexity metric may include thematic development which is the depth and intricacy with which topics or themes are explored. In some embodiments, the discourse complexity metric may include argumentative structure which is the presentation and support of claims, counterarguments and resolutions.

In some embodiments, the discourse complexity metric may include a comparison of implicit and explicit information, that is, the balance between what is directly stated and what is implied or left unsaid. In some embodiments, the discourse complexity metric may include interactivity which is a measure of the level of engagement and interaction between speaker and listener or writer and reader. In some embodiments, the discourse complexity metric may include intertextuality which refers to references to other texts or discourses within a given discourse. In some embodiments, the discourse complexity metric may include modality and modulation, which is the use of linguistic resources to express likelihood, necessity, obligation, or evaluation. In some embodiments, the discourse complexity metric may include pragmatic factors such as the consideration of context, speaker intention, and listener/reader interpretation.

The brain health determination module 272, is configured to receive as inputs the speech quality dataset and the speech content dataset and determine a speech score indicative of the participant's speech in relation to any existence and/or progression of a neurological condition and/or the status and/or changes in or status of a participant's brain health. The speech score may comprise one or more composite scores such as a: communication effectiveness score, dysarthria score, disease severity score, social communication score, voice quality score, intelligibility score and/or naturalness score. In some embodiments, the speech score may indicate the severity of a known neurological disorder and/or a participant's brain health, compared to a statistically, or experimentally determined scale. In some embodiments, the speech score may indicate a progression of an existing neurological disorder or a known brain health issue/attribute, specific to a particular participant. In some embodiments, the speech score may be determined, or partially determined, using previous speech scores. The previous speech scores may be from one or more participants.

In some embodiments, the intelligibility score may be formed from a speech intelligibility metric. The intelligibility score may provide an indication which relates to the clarity of speech, that is, how clearly a speaker speaks so that the speech is comprehensible to a listener. In some embodiments, the intelligibility score may be formed from intrusive intelligibility metrics and non-intrusive intelligibility metrics. In some embodiments, the intelligibility score may take into account intelligibility metrics, including articulation index, speech-transmission index and coherence-based intelligibility.

In some embodiments, the naturalness score may also be referred to as smoothness, healthiness, or bizarreness. In reference to speech, naturalness pertains to the degree to which spoken language sounds fluid, effortless, and typical of a human speaker. Naturalness indicates a lack of artificiality, affectation, or awkwardness. When evaluating speech synthesis systems, like text-to-speech engines, naturalness is a key criterion, reflecting how closely the synthetic speech mirrors genuine human speech in terms of intonation, rhythm, stress, and other prosodic features. In some embodiments, the naturalness score is calculated by differentially weighting distinct components of speech that span the speech subsystems. The naturalness score may be achieved through use of machine learning and/or regression models. In some embodiments, the machine learning model may be a supervised or unsupervised model. In some embodiments, the regression model used may include, but is not limited to, linear regression, logistic regression, polynomial regression, stepwise regression, Bayesian linear regression, quantile regression, principal components regression, Elastic net regression, ridge regression, lasso regression. The naturalness score which is formed from these components may be referred to as naturalness composites. In some embodiments, supervised statistic machine learning frameworks are utilised to maximise the transparency of included features. These distinct components which form the naturalness score may include, but are not limited to, respiration, phonation (voice quality), articulation, resonance, and prosody. In some embodiments, naturalness composites may include measures of prosody, in which the rhythm, stress, and intonation patterns are examined. The distribution and pattern of stressed syllables, as well as the overall prosodic contour of utterances, contribute significantly to the perception of naturalness.

Natural speech has variation in pitch, and the naturalness score may include measures of fundamental frequency (F0) variability, in which the mean, standard deviation, and contours of the fundamental frequency are measured to evaluate its variation and patterns. Natural speech will also have a dynamic intensity, and the naturalness score may include measures of intensity (or loudness) variability, in which the average intensity, its variability, and patterns are measured over time. In some embodiments, the naturalness score may further include measures of formant transitions, in which rapid movements in the formant frequencies (especially F1 and F2) indicate fluid transitions between vowels and consonants. Speech rate & rhythm may also form part of the naturalness score, where the number of syllables or words per unit of time is calculated. Additionally, examining the variability in duration of vowels, consonants, and pauses can provide insights into the rhythm of speech, and be used as a measure of speech rate and rhythm.

In some embodiments, the measures may be limited by upper or lower thresholds. Some thresholds may be dictated by basic anatomy, for example, during typical conversation adult voices don't often go outside the range of 50 Hz to 500 Hz. Deviations in measures which exceed predetermined thresholds may be used to change the weighting of the measure in determining the naturalness score. In some embodiments, deviations from a threshold may result in the measure being weighted more or less in determining the naturalness score. In some embodiments, deviations may be used to determine potential errors or low quality in the sound and/or text being analysed.

In further embodiments, the naturalness score may include measures of phonation, or voice quality, where parameters like frequency perturbation (also called jitter) and amplitude perturbation (also called shimmer) can be assessed. High values of frequency perturbation and amplitude perturbation might indicate voice disorders or result in a finding of unnaturalness (or a low naturalness score). Further, spectral measures may also be included in the naturalness score. In spectral measures, the Harmonic-to-Noise Ratio (HNR) can be used to determine the amount of noise in the speech signal. A lower HNR might indicate breathiness or hoarseness, potentially making speech sound less natural (and resulting in a lower naturalness score). The naturalness score may additionally include measures of resonance.

In some embodiments, the naturalness score may further include temporal measures, where the duration of segments, pauses and the rate of speech as investigated and analysed. Measures of articulation and coarticulation, including examination of how sounds influence one another, may also be included in the naturalness sore. Natural speech will have a degree of overlap and blending between adjacent sounds. In some embodiments, the naturalness score, or naturalness composites may be formed, created or generated from one or more metrics including: fundamental frequency variability, intensity variability, formant transitions, speech rate and rhythm, phonation measures, spectral measures, temporal measures, articulation, coarticulation, resonance and prosody.

The condition determination module 274, is configured to receive the speech score and determine the presence, progression or regression of a neurological disorder and/or brain health. The determination may comprise a list of possible neurological disorders that may require further testing, an ordered list comprising probabilities/likelihood of the participant being afflicted by one or more neurological disorder and/or a yes/no determination of the presence of a particular neurological disorder or brain health condition. In some embodiments, the participant may already be diagnosed with a neurological disorder and/or have known brain health issues, and the system 200 may be aware of this either from previous speech tasks and/or recording tasks, or from medical data entered as part of the participation and/or sign up process. In the case that the participant has an existing disorder, the condition determination module 274 may be configured to compare previous speech scores to the presently determined speech score and determine a progression of the existing disorder.

Speech analysis server may comprise or otherwise make use of AI models that incorporate deep learning based computation structures, including artificial neural networks (ANNs). ANNs are computation structures inspired by biological neural networks and comprise one or more layers of artificial neurons configured or trained to process information. Each artificial neuron comprises one or more inputs and an activation function for processing the received inputs to generate one or more outputs. The outputs of each layer of neurons are connected to a subsequent layer of neurons using links. Each link may have a defined numeric weight which determines the strength of a link as information progresses through several layers of an ANN. In a training phase, the various weights and other parameters defining an ANN are optimised to obtain a trained ANN using inputs and known outputs for the inputs. The optimisation may occur through various optimisation processes, including back propagation. ANNs incorporating deep learning techniques comprise several hidden layers of neurons between a first input layer and a final output layer. The several hidden layers of neurons allow the ANN to model complex information processing tasks, including the tasks of determining standard and non-standard user behaviour performed by the system 200.

In some embodiments, the ML model may incorporate one or more variants of convolutional neural networks (CNNs), a class of deep neural networks to perform the various processing operations for determining brain heath. CNNs comprise various hidden layers of neurons between an input layer and an output layer that convolve an input to produce the output through the various hidden layers of neurons.

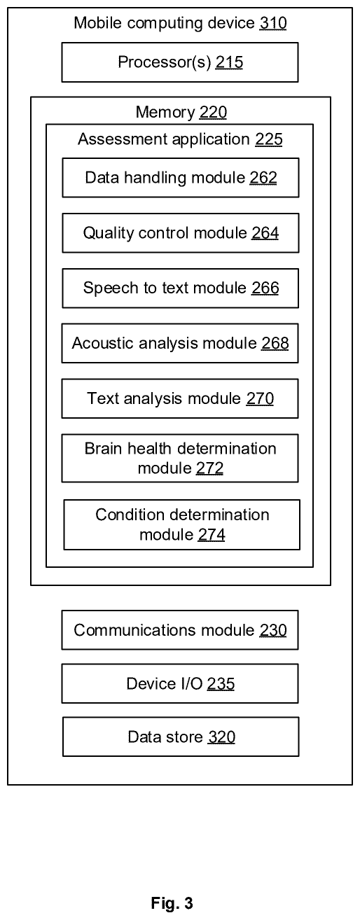

FIG. 3 is a block diagram of mobile computing device 310 for determining brain health, according to some embodiments. Mobile computing device 310 may comprise the same or similar components as audio analysis server 250 required to perform the disclosed methods. The client device 310 may be an ‘all-in-one’ system, where assessment application 225 comprises data handling module 262, quality control module 264, speech to text module 266, acoustic analysis module 268, text analysis module 270, brain health determination module 272 and/or condition determination module 274 as described above with reference to FIG. 2. Client device 310 may also comprise data store 320, which may be used to store recorded audio that is yet to be processed, and/or brain health determinations and data associated with brain health determinations that have been performed by mobile computing device 310. Client device 310 may also be in communication, via communications module 230 and/or network 240 with database 245. In some embodiments, client device 310 may be configured to perform all processing steps to perform the present disclosures.

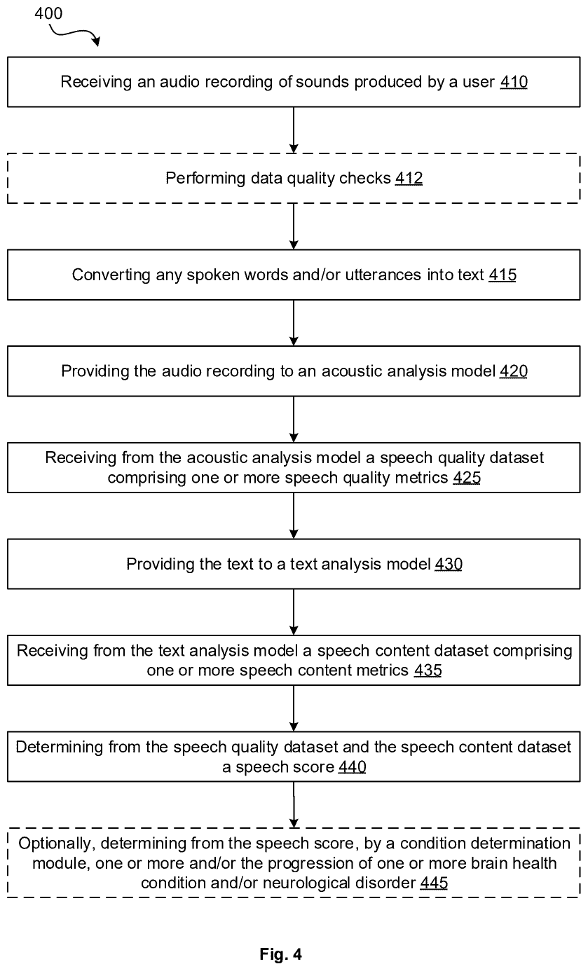

FIG. 4 is a process flow diagram of a method 400 of determining brain health, according to some embodiments. Method 400 may be performed by the embodiment of system 200 or the embodiment of system 300, or any other configuration of hardware and/or software, installed and/or executed on one or more server, system and/or client device. The steps of method 400 as depicted in FIG. 4 and described below may correspond more or less to the steps of method 100 as described above and in FIG. 1. In some embodiments, one or more steps of method 400 may correspond to the same step of method 100.

At 410, data handling module 262 receives an audio recording of a user or participant speaking. In some embodiments, Step 410 may correspond with step 1 of FIG. 1. The audio recording may be a .WAV file, a .PCM file, an .AIFF file, a .MP3 file, or .WMA format, in some embodiments. The audio recordings may be lossy or lossless. The audio recording may be a pre-recorded audio recording that has been stored in and transferred from database 245, or the audio recording may have been recorded by and subsequently communicated from mobile computing device 210. In some embodiments, the audio recording may be recorded and communicated in real time (i.e. streamed) from the mobile computing device 210 to audio analysis server 250.

In some embodiments, data handling module 262 may receive two or more audio recordings at a time. In this instance, data handling module 262 may be configured to temporarily store the two or more audio recordings, and transmit each recording to other modules in the audio analysis server 250 as required. For example, if a participant completes three speech tasks and/or extended audio recordings as part of their assessment, data handling module 262 may receive all three recordings in quick succession. Data handling module 262 may communicate any one of the three audio recordings to one or more modules of memory 260, and store the remaining two audio recordings. In some embodiments, the data handling module 262 may communicate one of the two stored audio recordings after a predetermined period of time, or it may transmit one of the two stored audio recordings after receiving an indication from one or more modules of memory 260, indicating that the one or more modules are ready to receive another audio recording.

At optional step 412, quality control module 264 performs data quality checks to determine if the audio recordings are suitable for use in determining/assessing brain health. In some embodiments, optional step 412 may correspond with step 1A of FIG. 1. The quality control process may comprise determining if the audio recording is suitable for use in determining brain health by conducting one or more quality tests on the audio recording. The quality tests may include, but may not be limited to: checking if the sound file is empty; detecting whether other speakers additional to the primary speaker have been recorded; checking stimuli produced aligns with target stimuli according to one or more selected task and the associated minimum requirements; and/or checking a signal to noise ratio of the recording. The quality checks may also comprise any one or more, or any combination of two or more of determining if a voice is present, determining a recording duration, determining the recording duration is within a predetermined recording limit, remove aberrant noise; remove silence; determine the number of recorded speakers, separate speakers.

In some embodiments, at optional step 412, the one or more quality tests may each have its own pass threshold that is indicative of an acceptable level of the quality that test is indicative of. Some quality tests may be binary pass/fail tests, such as whether the audio file is empty or not. For some quality tests, the pass threshold may be a proportion of the recording that contains a certain unwanted quality, for example, an audio recording may pass a quality test if the signal to noise ratio is less than 50%, for example. Other quality tests may have pass thresholds related to the intensity of a particular feature of the audio recording, for example, if a particular quality test is assessing the presence of two or more speakers, if the additional speaker's voice does not reach a minimum decibel rating, the audio recording may fail that particular quality test.

In some embodiments, the quality control module 264, at optional step 412, may also conduct one or more quality improvement processes, including but not limited to: filtering out background noise such as clicks, static and/or audio artefacts; where voice intensity is concerned, equalising speech intensity to assist analysis of things such as syllable emphasis; and/or clipping periods of extended silence from the audio file, such as large pauses in speech and silence at the beginning and end of the recording.

In some embodiments, if the quality control module 264 determines that the audio recording is not of sufficient quality, for example by the audio recording failing to pass (e.g. not meeting the pass threshold) of a certain number of quality checks, the quality control module may be configured to mark the particular audio recording as not suitable. In some embodiments, when an audio recording is determined to not be suitable, the quality control module 264 may communicate a notification/indication to the mobile computing device 210, indicating that a particular audio recording is of unacceptable quality. In some embodiments, the notification/indication may also include a prompt to re-record the particular audio recording by redoing the associated speech task, or recommencing/restarting the extended recording of audio.

At 415, speech to text module 266 receives from either data handling module 262 or quality control module 264 the audio recording. In some embodiments, step 415 may correspond with step 2 of FIG. 1. The speech to text module 315 is configured to analyse the audio recording and generate a textual representation of the spoken words as recorded by the speech recording. For example, the data handling module will receive as an input an audio file of a recording of speech, and output a file that includes text, such as a .docx, .PDF, and/or .txt. The speech as defined by the audio recording may be turned into text via the use of a machine learning model trained to accept an audio recording as an input.

In some embodiments, the speech to text module 266 comprises a speech to text model, configured to receive a representation of the audio recording, such as a sequence of filter bank spectra features, or a sonic spectrogram, for example, and output a string of text, indicative of the words spoken in the audio recording.

In some embodiments, the speech to text module 266 may first convert a received audio file into a sequence of filter bank spectra features. A filter bank is an array of bandpass filters that separates the input signal into multiple component frequencies, each one carrying a single frequency sub-band of the original signal. The sequence of filter bank spectra features is a representation of the frequencies present in the audio file.

The speech to text module 266 may comprise a speech to text model, trained and configured to receive the sequence of filter bank spectra features and output a textural representation of the sounds made by the participant.

The speech to text model may comprise one or more artificial neural networks (ANNs) to accomplish the task of converting spoken language into text. ANNs are computation structures inspired by biological neural networks and comprise one or more layers of artificial neurons configured or trained to process information. Each artificial neuron comprises one or more inputs and an activation function for processing the received inputs to generate one or more outputs. The outputs of each layer of neurons are connected to a subsequent layer of neurons using links. Each link may have a defined numeric weight which determines the strength of a link as information progresses through several layers of an ANN. In a training phase, the various weights and other parameters defining an ANN are optimised to obtain a trained ANN using inputs and known outputs for the inputs. The optimisation may occur through various optimisation processes, including back propagation. ANNs incorporating deep learning techniques comprise several hidden layers of neurons between a first input layer and a final output layer. The several hidden layers of neurons allow the ANN to model complex information processing tasks, including the tasks of speech analysis and text generation performed by the system 200.

In some embodiments, the speech to text model may comprise an encoder recurrent neural network (RNN), configured to listen, and a decoder RNN, configured to spell. An RNN is a particular type of ANN where connections between neurons form a directed or undirected graph along a temporal sequence. RNNs are capable of exhibiting temporal dynamic behaviour, i.e. they may process inputs using time as a contributing factor to the eventual determination of an input. This makes RNNs well suited to performing speech recognition. The encoder and decoder RNNs may be trained jointly, at the same time with the same training dataset.

The listener RNN accepts as inputs the sequence of filter bank spectra features, which are transformed via the layers of the RNN into higher level features. Higher level features may be shorter vocal sequences, such as individual phonemes and/or various versions of individual phonemes depending on their location in a word, and/or surrounding phonemes, for example. Phonemes are the smallest unit of speech distinguishing one word (or word element) from another. Once the listener RNN has processed the input and determined the sequence of high level features, the listener RNN may then provide the determined higher level features to the decoder RNN.

The decoder RNN spells out the spoken audio into a written sentence one letter at a time. To determine the initial or next word in the sounds made by the participant, the decoder RNN may produce a probability distribution conditioned on all the characters seen previously. The probability distribution for the next character to be determined is a function of the current state of the decoder RNN and the current context. The current state of the decoder RNN is a function of the previous state, the previously emitted character and the context. The context is represented by a context vector, which is produced by an attention mechanism.

At each time step through the speech to text determination process, the attention mechanism generates the context vector, which encapsulates the information contained within the acoustic signal (i.e. the sounds made by the participant), which is needed to determine the next character. Specifically, at each time step, the attention context function computes the scalar energy for each time step. The scalar energy is converted into a probability distribution of the next character over times steps (or attention) using a normalisation function, such as the softmax function.

The final output of the speech to text model may be determined by using a left-to-right beam search algorithm. At each time step in the determination process each partial hypothesis in the beam is expanded with every possible character and only the most likely beams are kept. When an end of sentence token is encountered, it is removed from the beam and added to the set of complete hypotheses, thus generating, in a step-wise fashion, the textural representation of the sounds made by the participant.

In some embodiments, the speech to text model may determine that a textual representation of the audio recording cannot be determined. The speech to text model may determine that the audio recording does not comprise any sounds that can be represented textually, for example, the audio recording may not comprise any morphemes that can be represented by text, such as ‘er’, ‘ly’, ‘ish’ or ‘ic’. In some embodiments, the audio recording may comprise bodily sounds, such as gurgling, coughing, breathing, wheezing and/or heaving, which the speech to text model may not represent textually. In some embodiments, the speech to text model may represent bodily sounds by labelling their occurrence, for exampling indicating that the participant has coughed by generating text such as “[coughing]”, “*coughing*”, “{coughing}”, wherein the use of brackets, asterisks and/or any other symbol may indicate the occurrence of the bodily sound. In some embodiments, the speech to text model may generate one or more descriptive sentences indicating the one or more bodily sound(s) that were recorded.

In some embodiments, if the speech to text model determines that no textual representation can and/or should be determined, the step of generating a textual representation may be skipped. In some embodiments, determining that no textual representation should be generated may comprise determining that the audio recording does not contain at least a single morpheme, phoneme, or sound that can be represented textually. Subsequent to determining that no textual representation can and/or should be determined, the speech to text module 266 may generate an indication indicative that no textual representation was created. In some embodiments, the indication indicative that no textual representation was created may be sent to or otherwise communicated to the text analysis module 270 for use in the method 400.

In some embodiments, the method 400 may not comprise step 415 of converting a recording of sounds of the participant into a textual representation and/or determining whether a textual representation is able to be and/or required to be generated and/or determined.

At 420, the acoustic analysis module 268 receives the audio recording from data handling module 262 or quality control module 264. In some embodiments, step 420 may correspond with step 3B of FIG. 1. The acoustic analysis module may process the audio recording to determine a dataset of acoustic analysis metrics.

The acoustic analysis module 268 may process the audio recording using a machine learning model. The machine learning model may be trained using a dataset of fully labelled, partially labelled or unlabelled data. The data may be audio recordings of speech. In some embodiments, the audio records may be of participants completing one or more speech task and/or one or more extended audio recording of the participant. The ML model may be trained iteratively on the training data set, and tested using a validation dataset. In some embodiments, the training and validation datasets may be subsets of a larger dataset, and sampled randomly to generate multiple testing and validation datasets.

In some embodiments, the acoustic analysis module 268 may comprise an acoustic analysis model configured to perform acoustic analysis and categorisation of the input. In some embodiments the acoustic analysis model may be a trained RNN, trained or otherwise configured to receive an audio file, or a representation of an audio file, such as a sonic spectrogram, wave form representation, filter bank spectra features, and/or numerical encoding. In some embodiments, each audio file may be converted into a time-series wave form representation, this time series wave form representation may then be decomposed into a set of specific frequencies and/or frequency bands. In some embodiments, the time series wave form representation and or set of specific frequencies and/or frequency bands may be an image file, such as a .JPEG, .BMP, or .PDF, for example. This set of frequencies and/or frequency bands may then be provided as a multi-variant input to one or more ML models.

The acoustic analysis model may process the input and return a categorisation, or other determination of one or more qualities of the input audio file or audio file representation. Qualities may include prosody and timing, articulation, resonance, and/or quality, for example.

In some embodiments, to perform the acoustic analysis, the acoustic analysis module 268 may use Bayesian statistical models along with deep learning and recurrent neural networks (RNN) to incorporate the time course nature of progression or change. For example, Long Short-Term Memory networks are a special class of RNNs that outperform many other previous forms of machine learning and deep learning architectures for tracking time-course data. In some embodiments, measures of variable importance may be obtained to provide parsimonious and interpretable models, thus allowing for the effect of interventions/progression to be inferred. Output from this stage of the analysis will be interpreted in the context of clinical data (e.g. disease severity, cognition, fatigue).

In some embodiments, the acoustic analysis module 268 may comprise multiple ML models, each model having been trained on a specific dataset, each dataset configured to represent a particular brain health attribute, neurological disorder and/or brain health metric. In some embodiments, where two or more brain health attributes, neurological disorders and/or brain health metrics often co-occur and/or share similar acoustic signatures and/or attributes, one of the multiple models may be trained or otherwise configured to categorise two or more brain health attributes, neurological disorders and/or brain health metrics.

In some embodiments, the acoustic analysis module 268 may comprise multiple speech quality ML models, each speech quality model trained to categorise a particular speech quality. In some embodiments, the acoustic analysis module 268 may also comprise a ML model that may accept as inputs, determinations by the multiple ML models configured to categorise a particular speech quality. For example, the multiple ML models may return distinct indications of two or more of the prosody and timing, articulation, resonance, and/or quality of the input audio representation. The distinct indications may be a numerical representation on a predetermined scale, or a categorisation of a particular brain health condition, or neurological condition. The predetermined scale may be experimentally determined, or it may be determined based on previous test results associated with the present participant.

The distinct indications may be provided to a summation ML model, which may provide a summary categorisation of the audio input. The summary categorisation may be a numerical representation of the participant's brain health. For example the summary categorisation may be a number on a predetermined scale, the predetermined scale having been experimentally determined, or having been determined based on previous test results associated with the participant.

In some embodiments, the training of one or more acoustic analysis models may comprise a sliding window data selection approach, to control for one or more brain health attributes, neurological disorders and/or brain health metrics. The sliding window may assess each input during the training process to determine its suitability, relevance and/or relatedness to previous and/or subsequent inputs. For example, the sliding window may determine that an audio input is indicative of a particular brain health attribute, neurological disorder and/or brain health metric. The sliding window may then curate subsequent inputs that are also indicative of the same and or related brain health attribute, neurological disorder and/or brain health metric.

In some embodiments, the training process may comprise a semi-supervised training approach. The semi-supervised training approach may comprise using a dataset of both labelled and unlabelled data. For example, the training dataset may comprise a small number of labelled data and a large number of unlabelled data, such as a relatively small number of abnormal brain health recordings labelled as being indicative of altered brain health. The training dataset may also comprise a large number of unlabelled data, which may contain both normal and abnormal brain health data, but with no associated tag/label.

In some embodiments, the semi-supervised training approach may be a self-training approach, wherein an initial ML model is trained on the small collection of labelled data to create a first classifier, or base model. The first classifier may then be tasked with labelling one or more larger unlabelled datasets to create a collection of pseudo-labels for the unlabelled dataset. The labelled dataset is then combined with a selection of the most confident pseudo-labels from the pseudo-labelled dataset to create a new fully-labelled dataset. The most confident pseudo-labels may be hand selected, or determined by the ML model. The new fully-labelled dataset is then used to train a second classifier, which by nature of having a larger labelled training dataset may exhibit improved classification performance compared to the first model. The above-described process may be repeated any number of times, with more times generally resulting in a better performing classifier.

In some embodiments, the semi-supervised training approach may be a co-training approach, wherein two first classifiers are initially trained simultaneously on two different labelled data sets or ‘views’, each labelled data set comprising different features of the same instances. For example, one dataset may comprise a frequency set below a certain threshold, and one may comprise a frequency set above a certain threshold. In this approach each set of features is sufficient for each classifier to reliably determine the class of each instance.

Subsequent to the initial training of the two first classifiers, the larger pool of unlabelled data may be separated into the two different views and given to the first classifiers to receive pseudo-labels. Classifiers co-train one another using pseudo-labels with the highest confidence level. If the first classifier confidently predicts the genuine label for a data sample while the other one makes a prediction error, then the data with the confident pseudo-labels assigned by the first classifier updates the second classifier and vice-versa. Finally, the predictions are combined from the updated classifiers to get one classification result. As with the self-training approach, this process may be repeated iteratively to improve classification performance.

In some embodiments, training the ML model may use a deep generative model to compensate for the imbalance between normal and abnormal brain health data. Generative models treat the semi-supervised learning problem as a specialised missing data imputation task for the classification problem, effectively treating data imbalance as a classification issue instead of an input issue. Generative models utilise a probability distribution that may determine the probability of an observable trait, given a target determination. Generative models have the capability to generate new data instances based upon previous data instances, to aid in training better performing models for datasets with limited labels.

The use of semi-supervised learning models may be used to account for the lack of certain types of data pertaining to certain brain health conditions. For example, audio data from participants with brain cancer may be sparse, so the above-described semi-supervised methods may help to create more reliable models. In another instance, two or more brain health conditions may appear to present in extremely similar ways in terms of speech, and accordingly models may have trouble reliably determining the difference. Semi-supervised learning may be used to increase the accuracy of one or more models at reliably classifying one condition from the other.

In some embodiments, the acoustic analysis model may comprise an information sieve. The information sieve may be configured to perform a hierarchical decomposition of information that is passed to it. The information sieve may comprise multiple, progressively fine-grained sieves. Each level of sieve may be configured to recover a single latent feature that is maximally informative about multivariate dependence in the data. In some embodiments, a representation of an audio recording of a participant may be provided to the sieve. The sieve may extract certain qualities associated with the representation that have been determined to be indicative of a particular brain health attribute, neurological disorder and/or brain health metric. In some embodiments, each layer of the information sieve may be configured to extract a particular indicator and/or feature of the audio representation that is associated with a particular speech characteristic. The sieve may return each of the latent features in a latent features dataset. In some embodiments, the information sieve may be configured to assess the latent features and return a determination regarding the particular speech characteristic being assessed.

Some embodiments may comprise two or more information sieves, each being configured to assess a particular brain health condition and/or speech characteristic. Each determination received from each information sieve may be used as an input to determine a participant's brain health. In some embodiments, the acoustic analysis model may use each of the determinations from each of the information sieves to determine a final speech determination.

In some embodiments, the training data used to train the one or more ML models or artificial intelligence models may comprise one or more data curation procedures and/or one or more data preparation procedures. The one or more data curation procedures and/or one or more data preparation procedures may comprise the removal of low quality or incorrect data. It may also comprise correction of incorrect or poor quality data. The one or more processes may also comprise relabelling of incorrectly labelled data. The one or more data curation procedures and/or one or more data preparation procedures may further comprise labelling of unlabelled data. They may also comprise the removal of unwanted portions of data, while leaving the remaining, useful data intact.

At 425, the acoustic analysis model outputs a determination of the quality and/or characteristics of the recorded sound. In some embodiments, step 425 may correspond with step 3B of FIG. 1. In some embodiments, the determination may be a speech quality dataset, comprising one or more speech quality metrics. The speech quality metrics may be one or more of: prosody and timing, articulation, resonance, and/or quality.

Prosody features include measures at the individual sounds or iteration level relating to variations in timing, stress, rhythm, intonation, and stress of speech. These could include actual and variance of amplitude, frequency and energy dynamics. The prosodic features may be determined at words/intra word intervals, language formation/breath, across conversations between speakers, as episodes of stuttering, blocks/prolongations, syllabic rate, articulation rate, or across continuous recording periods. Articulation may be determined using the frequency distribution of the speech signal. These may include Power Spectral Density (PSD) and Mel Frequency Cepstral Features (MFCCs), formant slope, voice onset time and/or vowel articulation scores. Resonance may be determined using power and energy distribution features such as MFCCs, octave ratios, frequency/intensity threshold and/or formants. Quality may be determined using source features designed to measure VF vibratory patterns, aperiodicity and/or aerodynamics.

At 430, the textual representation of the sounds made by the participant is provided to the text analysis module 270. In some embodiments, step 430 may correspond with step 3A of FIG. 1. In some embodiments, text analysis module 270 may comprise a text analysis model. The text analysis model may be a machine learning model configured to perform natural language processing (NLP). In some embodiments, an ANN may be trained using a dataset of text. The text analysis model 170 may utilise word embeddings to represent relationships between words or sentences in n-dimensional space. The word embeddings may be in the form of a real-valued vector that encodes the meaning of the word such that the words that are closer in the vector space are expected to be similar in meaning, use, content, and/or interpretation. The text analysis module may be configured to convert the textual representation into one or more word embeddings, which may then be given to the text analysis model, which may output a determination of one or more qualities of the text.

In some embodiments, the text analysis module 270 may receive the indication indicative that no textual representation was created from speech to text module 266.

At 435, the text analysis model returns a speech content dataset, comprising one or more speech content metrics. In some embodiments, step 435 may correspond with step 3A of FIG. 1. The speech content metrics may be indicative of one or more of: Lexico-semantic performance and verbal fluency, grammar and morphosyntactic performance, and/or discourse level performance (text production and comprehension).

Lexico-semantic performance may be determined by examining word retrieval, verbal knowledge, non-verbal semantic knowledge and comprehension and verbal fluency. Grammar and morphosyntactic performance may be determined by measuring sentence comprehension and grammar in challenge tasks and connected speech tasks/extended recordings. Verbal fluency may be determined by correctly spoken words/sentences, incorrectly spoken words/sentences, interjections and/or repeats. Lexical density may be determined by vocabulary range and/or ratio of types to tokens. Informational content may be determined by speech efficiency, semantic versus conceptual content, speech schemas, and/or cohesion. Grammatical complexity may be determined by morphological complexity, word classes, semantic complexity and/or subject-verb-object (SVO) order.

In some embodiments, the speech content metrics may be numerical values that indicate a level of competence, performance, execution or other measurement on a scale related to each metric. For example, one of the metrics may be indicative of verbal fluency on a scale from 0 to 100, 0 being associated with no verbal fluency at all and 100 being associated with exceptional verbal fluency. A textual representation of spoken words by a participant may, for example, be determined as 50/100 on the verbal fluency scale. This may be indicative of a participant having average verbal fluency compared to similar participants.

In some embodiments, the scale may be experimentally determined from collecting data on a plurality of participants. The scale may also be a personalised scale that is indicative of a single participant's previous performance. In some embodiments, the metric may be an indication of whether a participant has performed better or worse than one or more previous recordings.

In some embodiments, the text analysis module 270 may output a single metric indicative of the participant's speech content, as determined via the textual representation of any words spoken by the participant during the recording. This single metric may be indicative of one or more of: the participant's speech content generally, for example, compared to other similar participants; whether the participant's speech content is particularly indicative of one or more specific brain health conditions, neurological conditions and/or disabilities; and/or an indication of improvement or deterioration of the participant's speech content.

In some embodiments, the text analysis module 270, subsequent to receiving the indication indicative that no textual representation was created, may determine and/or generate a speech content dataset, comprising one or more speech content metrics that is indicative of the fact that the participant did not make any sounds that were able to be, and/or deemed necessary to convert from audio into a textual representation.

In some embodiments, subsequent to step 415 not being performed (i.e. not generating a textual representation and/or determining whether a textual representation is able to be and/or required to be generated and/or determined) steps 430 of providing the textual representation to the text analysis module 270 and step 435, of determining a text content dataset may not be performed. In some embodiments, a textual representation may still be determined by speech to text module 266, but the textual representation may not be supplied to text analysis module 270 and text analysis module 270 may not determine a speech content dataset. Text analysis module may receive a textual representation, or indication that no textual representation was able to be determined, but may not determine a speech context dataset, in some embodiments.

In some embodiments, such as depicted in FIG. 1, steps 415, 420, 425, 430 and/or 435 may be performed in sequence, parallel, synchronously (i.e. as part of a single series of steps and/or actions performed or taken one after the other or in parallel) and/or a synchronously (i.e. performed separately, at substantially different times, wherein the results may be stored for later and/or subsequent use). In some embodiments, the steps of converting any spoken words and/or utterances in text 415, providing the text to a text analysis model 430, and receiving from the text analysis model a speech content dataset comprising one or more speech content metrics 435 may be performed in sequence but in parallel with the steps of providing the audio recording to the acoustic analysis model 420 and receiving from the acoustic analysis model a speech quality dataset comprising one or more speech quality metrics 425.

In some embodiments, steps 415, 420, 425, 430 and/or 435 may be performed sequentially, in the order depicted in FIG. 4, or in any other order that would eventuate in the performance of a method of determining brain health, according to the present disclosures.

In some embodiments, if the speech to text model has not determined a textual representation, the speech to text model may generate a speech content dataset that is indicative of the lack of any recorded sounds that can be represented textually. In some embodiments, the speech content dataset may comprise indications that bodily sounds have been recorded, but are not capable of being textually represented.

At 440, the brain health determination module 272 may receive the speech quality dataset and the speech content dataset and determine a speech score. In some embodiments, step 440 may correspond with step 4 of FIG. 1. The speech score may be indicative of the brain health of the participant. The speech score may indicate the presence of a neurological condition of the participant, or the status of the participant's brain health when no neurological condition was known previously (i.e. a recent onset of a neurological condition/deterioration of brain health). In some embodiments, the speech score may be indicative of the progress of a known neurological condition, and/or changes in a participant's brain health. The progression of the known neurological condition and/or changes in brain health may be determined by comparing the newly determined speech score with one or more previously determined speech scores associated with the participant.

The speech score may comprise one or more composite scores, indicative of various speech characteristics. The composite scores may be one or more of a communication effectiveness score, dysarthria score, disease severity score, social communication score, voice quality score, intelligibility score, and/or naturalness score.

In some embodiments, at 445 the brain health determination module 272 may comprise one or more brain health models. In some embodiments, the brain health model may be a type or variant of a NN, trained or otherwise configured to receive as inputs scores, metrics, or other determinations of speech content and/or speech quality, generated, classified, calculated or other otherwise determined by one or more of the acoustic analysis module 268 and/or the text analysis module 270 and to output a determination of brain health. The brain health determination may be a set of metrics that are indicative of brain health, and/or progression/existence of one or more neurological conditions. In some embodiments, the determination may be a classification as to the existence and/or progress of a known or suspected brain health condition and/or neurological disorder. The brain health NN may be configured with and/or otherwise comprise combinations of differentially weighted features indicative of brain health and/or aspects of brain health, derived and/or configured from/during the training process.

In some embodiments, the brain health model may derive one or more metrics and/or determinations indicative of and/or relating to brain health via a combination of attributes and/or features that map on to conditions, symptoms, disorders and/or indicators of brain health and/or neurological disorders. In some embodiments, the indicators of brain health may comprise listener ratings, speaker ratings, patient reported outcomes, and/or performance on another test.

In some embodiments, the brain health model may return a set of values, metrics or determinations, indicative of one or more multivariate participant brain health attributes, each of the brain health attributes being associated with one or more aspects of one or more of speech content and/or speech quality, as defined by the outputs of one or more of the speech quality model and/or speech content model as discussed above.

In some embodiments, each of the one or more brain health metrics may be a numerical value on a continuous scale. The continuous scale may be experimentally determined from data collected from a plurality of participants with or without brain health conditions or altered brain health. In some embodiments, the scale may be determined based on data associated with the particular participant, such as a participant's medical history; pre-testing of a participant before they begin and/or continue using the system 100 and/or previous determinations by the system 100.

In some embodiments, the brain health NN may be trained using a ground truth or other training dataset comprised on experimentally derived or determined data. The experimentally determined data may have be determined via disease severity scores determined through assessment by trained professionals; paper and pencil language tests devised by trained professionals and administered to participants, whose brain health conditions may or may not be known; clinical ratings or impressions collected as part of standard clinical operations and/or academic studies; and/or patient reported outcomes recorded such as via patient visits, surveys, clinical studies and/or routine doctor's appointments, for example.

In some embodiments, the brain health NN may utilise, comprise, incorporate and/or otherwise avail of any one or more of the training and/or data labelling, generation, and/or curation techniques/methods as discussed and/or referenced in this specification.

In some embodiments, certain aspects of the recorded sounds made by the participant and/or any textual representation thereof that are particularly predictive; and/or represent known or unknown correlations between metrics, symptoms and/or brain health conditions may be accounted for and/or incorporated into the disclosed methods. In some embodiments, considerations between efficiency, and accuracy may be made, as to assess the overall utility of one or more particular metrics and/or data types, or aspects of a data type, to ensure adequate processing/performance times.

In some embodiments, the speech score and/or brain health determination made by the brain health determination module 272 may be used by the condition determination module 274 to determination one or more brain health condition and/or neurological condition, and/or the progression of one or more brain health condition and/or neurological condition of a participant. This may be performed by comparing the newly determined speech score to one or more previous speech scores associated with the participant. In some embodiments, the condition may be determined by comparing one or more speech scores, including the newly determined speech score.

In some embodiments, the audio recording may be generated based on a task prompt for a speaker to perform a speaking task. A speaker may be presented with a speaking task, which is performed and recorded by an audio recording interface, such as device I/O 235. Mobile computing device 210 may automatically present to a user, or patient, a task prompt instructing them to speak out loud a particular phrase, sound or series of sounds. Each task may have a minimum requirement to be deemed as a successful attempt. In some embodiments, assessment application 225 may perform analysis on the audio recording to determine whether the minimum requirements have been met. In some embodiments, the mobile computing device may communicate the audio recording to the audio analysis server 250 without performing the analysis.

In some embodiments, during set-up and/or calibration of the present system, and/or during any point in the method 400 prior to determining the speech score (at 440), or any point prior to determining one or more and/or the progression of one or more brain health condition and/or neurological disorder (at 445), one or modules of the audio analysis server 250 may be configured to receive one or more narrowing parameters. The narrowing parameters may comprise, for example, one or more brain health conditions; one or more brain health attributes; one or more brain health metrics; one or more neurological disorders; and/or participant medical history. Upon receipt of one or more narrowing parameters, one or more models of the system 100 may be configured to adjust its determination process to address, incorporate and/or otherwise take account of the one or more narrowing parameters.

For example, the text analysis module 270 may receive a narrowing parameter that indicates the participant whose sounds are being recorded has a stutter. Accordingly, the text analysis module 270, upon receiving this narrowing parameter, may be configured to remove repeated utterances from the textual representation during the conversion from audio recording to text and/or after the conversion.

In another example, the brain health determination module 272 may receive a narrowing parameter that indicates the patient has one or more brain health diagnoses and/or conditions. In some embodiments, the brain health determination module 272 may be configured to adjust its determination to, rather than indicate the presence of the particular brain health diagnosis and/or condition, return a determination indicative of the severity and/or progress of the brain health diagnosis and/or condition.

In yet another exemplary embodiment, the acoustic analysis module 268 may receive a narrowing parameter that the participant is neurodivergent. The analysis module 268 may then be configured to adjust its determination to account for malformed sounds and/or mispronounced words, thereby returning a speech quality dataset and associated speech quality metrics that are indicative of the particular participants regular speaking habits.

In is noted that despite the fact that the tasks are referred to as speaking tasks, the participant may not form intelligible words, phrases, syllables, and/or sentences, for example when performing the task(s). However, this may not mean that the participant has not performed or otherwise participated in the speaking task. In other words, the speaking task is simply a prompt for the participant to make sounds that may be considered, attempt to speak, and/or otherwise use their vocal cords to produce some type of sound. In some embodiments, a speaking task may explicitly require the production of intelligible or otherwise understandable speech, or at least a portion of intelligible or otherwise understandable speech, such as a vowel sound (e.g. /ah/, /i/, /o/, /u/, /ae/, /e/), or other morphemes, and/or phonemes, for example.

The speaking tasks may comprise reading a set text, provided to the participant via a mobile computing device, such as a smart phone, laptop computer, desktop computer and/or kiosk. In some embodiments, the speaking task may be provided by one or more person's assisting the participant, and may be displayed on another separate mobile computing device or on a printed sheet. The minimum requirement of reading a set text may comprise the participant producing three seconds of audio recording containing speech.

The speaking tasks may also comprise performing unscripted contemporaneously produced speech on a random or specified topic. In some embodiments where the topic is specified, the topic may be given by the mobile computing device that is recording the sounds produced by the participant, such as via a screen/monitor and/or recorded audio queue. In some embodiments, one or more person's assisting the participant may provide the specified topic via a separate mobile computing device, oral instruction, and/or via a printed sheet. The minimum requirement may comprise three seconds of audio recording containing speech.

The speaking tasks may also comprise conducting a conversation with another speaker on a random or specified topic. In some embodiments where the topic is specified, the topic may be given by the mobile computing device that is recording the sounds produced by the participant, such as via a screen/monitor and/or recorded audio queue. In some embodiments, one or more person's assisting the participant may provide the specified topic via a separate mobile computing device, oral instruction, and/or via a printed sheet. The minimum requirement may comprise one exchange (i.e. one utterance for by each participant).

The speaking tasks may also comprise the production of a single vowel sound continuously (e.g. /a/, /i/, /o/, /u/, /ae/, /e/). The minimum requirement may comprise 0.01 seconds of audio recording containing sounds made by the participant.

In some embodiments, the participant may be recorded using a continuous recording protocol. Continuous recording protocols may be task agnostic and may capture biological sound (e. g breathing, coughing, vomiting) or as well as conversations, and/or one or more speaking tasks performed by the participant.

The speaking tasks may also comprise producing a single vowel sound continuously for as long as possible on one breath (e.g. /a/, /i/, /o/, /u/, /ae/, /e/). The participant may choose a vowel sound themselves, or be prompted to pronounce a particular vowel sound by the mobile computing device that is recording their speech, another mobile recording device, or by one or more person's assisting the participant, for example. The minimum requirement may comprise 0.01 seconds of audio recording containing speech.

The speaking tasks may also comprise producing alternating or sequential syllable strings repeatedly (e.g. pataka, patapata, papapapa, tatatata, kakakaka). In some embodiments, the participant may choose which alternating or sequential syllable strings themselves, or the participant may be given by the syllable string by the mobile computing device that is recording the sounds produced by the participant, such as via a screen/monitor and/or recorded audio queue. In some embodiments, one or more person's assisting the participant may provide a specific syllable string or a selection of syllable strings via a separate mobile computing device, oral instruction, and/or via a printed sheet. The minimum requirement may comprise one second of recorded audio containing speech.

The speaking tasks my also comprise saying the days of the week, in a particular order, in no particular order, starting a particular day of the week, or starting from an arbitrary day of the week. The minimum requirement may comprise one second of recorded audio containing sounds produced by the participant.

The speaking tasks may also comprise counting to a predetermined number or as high as possible. In some embodiments, the participant may choose which number they start counting from, which number they count to and/or a particular interval between a starting and ending number. In some embodiments, the participant may receive instructions as to a predetermined starting number, ending number and/or interval. The minimum requirements may comprise saying at least one number.

The speaking tasks may also comprise saying words following consonant vowel consonant structure (e.g. hard, heat, hurt, hoot, hit, hot, hat, head, hub, hoard). In some embodiments, the participant may be free to choose the particular words they pronounce that fit the consonant-vowel consonant structure, or the participant may be provided a list and/or sequence of words to recite. The minimum requirement may comprise one example of the consonant-vowel consonant structure.

The speaking tasks may also comprise repeating written or orally delivered phrases or sentences in varying length. In some embodiments, the written phrases or sentences may be presented via the screen/monitor of a mobile computing device, or presented on a written sheet of paper, for example. The minimum requirements may comprise the participant pronouncing at least one word.

The speaking tasks may also comprise repeating words with more than one syllable (e.g. computer, computer, computer). In some embodiments, the participant may choose a multi-syllabic word, be instructed to say a particular multi-syllabic word, or choose a multi-syllabic word from a predetermined list, for example. The minimum requirement may comprise the pronunciation of at least two iterations of the word.

The speaking tasks may also comprise, producing words where vowel transitions to produce more than one vowel sound (e.g. bay, pay, dye, tie, goat, coat). In some embodiments, the participant may choose their own words, be given a list of words to choose from, or a sequence of words to pronounce, for example. The minimum requirement may comprise pronunciation of at least one example.

The speaking tasks may also comprise repeating or reading a string of related words that increase in length and complexity (e.g. profit, profitable, profitability; thick, thicker, thickening). In some embodiments, the participant may choose their own words, be presented with particular words or given a list of words to choose from, for example. The minimum requirement may comprise the pronunciation of a single attempt of one word in the string.

The speaking tasks may also comprise verbally describing or writing down what the subject sees in an image, or what the subject sees around them. In some embodiments the image may be a single image or a series of images. In some embodiments, the participant may enter the description using one or more input devices, such as a keyboard, touchpad, mouse, dial, toggle, digital keyboard, onscreen keyboard and/or button, configured to interact with a mobile computing device, such as the mobile computing device that is performing recording of the sounds the participant is making, the mobile computing device that is determining the participant's brain health, or any other mobile computing device. In some embodiments, the participant may hand write their description and the handwriting may be manually entered, scanned or otherwise transferred into a digital form after the fact. The minimum requirement may comprise at least three seconds of audio recording containing sounds made by the participant, at least one distinguishable word pronounced by the participant, or at least one word written by hand and/or entered into a mobile computing device.

The speaking tasks may also comprise listening to or reading a story and then requiring the participant to retell what they heard or read. The participant may hear the story via a pre-recorded audio recording played back by a mobile computing device, such as at least one of the devices that are performing the brain health determination. In some embodiments, the participant may be read by one or more person(s) assisting them. In some embodiments, the participant may read the story from a computing device comprising a screen/monitor. The minimum requirement may comprise at least three seconds of audio recording containing speech or one word spoken by the participant.

The speaking tasks may also comprise generating a list of words within specific categories (e.g. words beginning with the letter F, or A, or S. words fitting within a semantic category, e.g. foods, or animals, or furniture). In some embodiments, the participant may be presented with a specific category or the participant may get to choose their own category, or select from a list of categories. The minimum requirements may comprise the pronunciation of one word.

The speaking tasks may comprise repeating made up words (e.g., yeecked or throofed). In some embodiments, the participant may make up their own made up words, or be presented with a list of made up words to recite. The minimum requirements may comprise the participant making a single attempt of one word.

The speaking tasks may comprise listening and repeating some words and/or sentences aloud, (e.g. single words/sentences spoken over noise such as cocktail noise, white noise). In some embodiments, a mobile computing device may play a recording comprising, for example the single words/sentences spoken over noise. The minimum requirements may comprise the pronunciation of a single attempt of one word in the string.

The speaking tasks may comprise describing what a viewer sees when looking at single item pictures (e.g. subject sees a picture of dog and says ‘dog’). In some embodiments, the participant may be presented with one or more pictures via a mobile computing device comprising a screen/monitor, or the participant may be presented with printed pictures featuring the single items from one or more person(s) assisting them. The minimum requirement may comprise a single attempt at identifying and pronouncing a single item.

In some embodiments, the minimum requirements may be configured to ensure that there is sufficient data that would allow the system 200 to perform the method of determining brain health according to any of the present disclosures. The minimum requirements may not be indicative of what a participant must do to be considered as satisfying or otherwise passing a speaking task. In some embodiments, a participant may provide the minimum required response, input and/or recording, but may still be deemed as not satisfactorily completing a speaking task. In other words, the minimum requirements of the recorded sounds of a participant may not be an indication of the participant's particular performance when undertaking one or more particular speaking tasks. In some embodiments, if a participant fails to meet the particular minimum requirements of one or more speaking tasks, this may be an indication that the participant as failed the one or more speaking tasks.

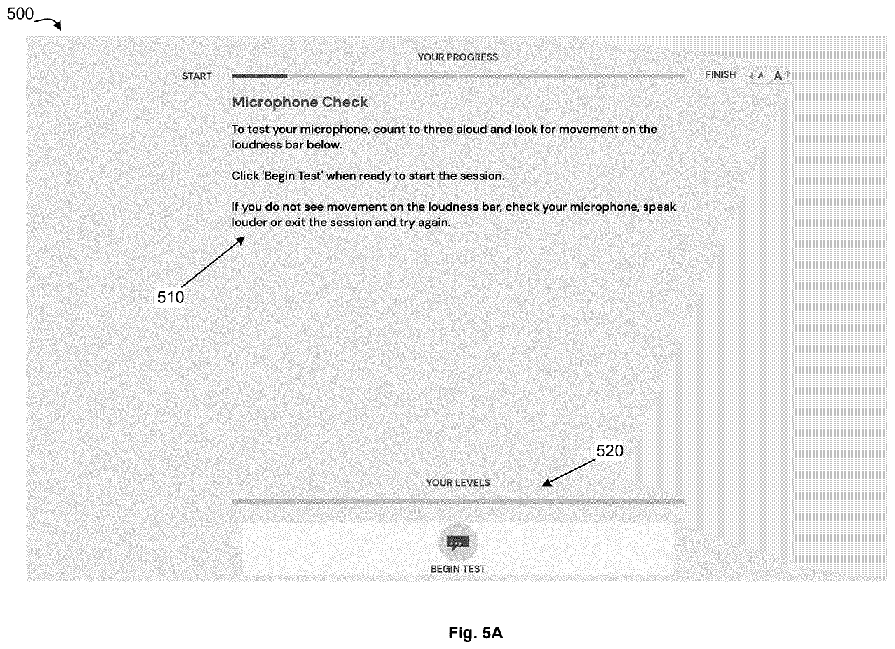

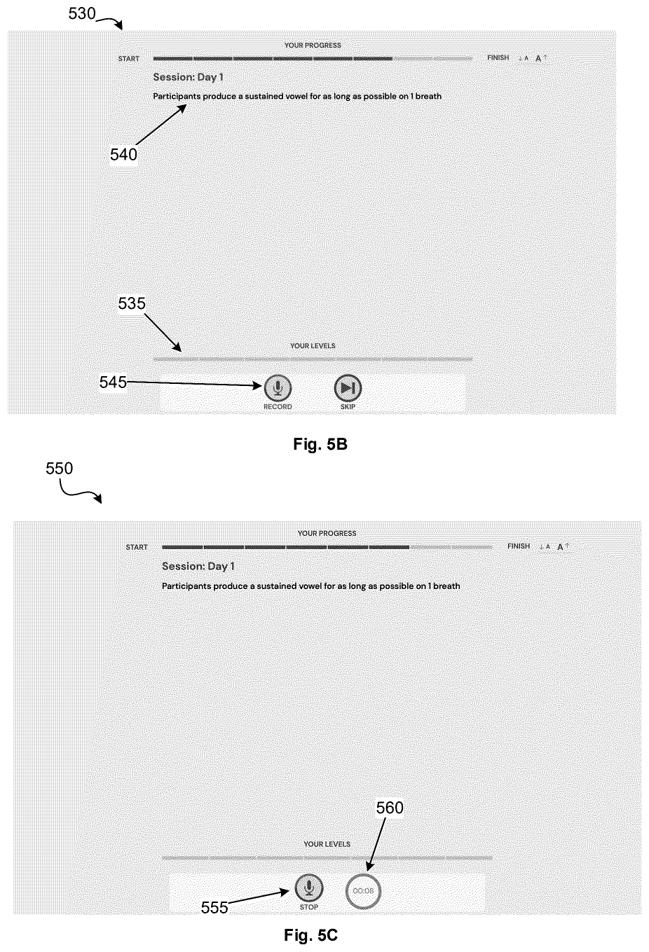

FIG. 5A is a screen shot 500 of a quality assurance check that may be implemented, according to some embodiments. The quality assurance check may be a microphone check, configured to determine whether the microphone that is being used to record sounds made by a participant is functioning properly. The quality assurance check screenshot 500 may comprise instructions 510, instructing the participant, and/or a user aiding the participant, the steps that must be taken to complete the quality assurance check. Screen shot 500 may also comprise quality assurance progress indicator 520, configured to indicate how far through and/or the success or failure of the quality assurance check. In some embodiments, the quality assurance check may be one or more of: a microphone check, to determine whether the microphone is responding to inputs; a level check, to determine whether inputs are within an acceptable decibel range; a network connectivity check, an available memory and/or storage check; and/or any other type of quality assurance check configured to ensure recording and/or processing proceeds as required.

FIG. 5B is a screen shot of a speech task screen 530, according to some embodiments. Speech task screen 530 is an example of a speech task screen corresponding to a speech task that is yet to be started and/or completed. In some embodiments, upon launching, starting, logging in or otherwise accessing assessment application 225, the participant and/or user aiding the participant may be presented with speech task screen 530. Speech task screen 530 may prompt the participant and/or user aiding the participant to complete a specific task. Speech task screen 530 may comprise task prompt 540, directing the participant to speak a certain phrase, make a certain sound, engage in a cognitive speaking task (e.g. say as many words that start with ‘H’) and/or any other type of speaking task. In some embodiments, task prompt 540 may direct the participant and/or us to begin recording for an extended period. Speech task screen 530 may also comprise task count 535, indicating the progress of the participant and/or user through a number of tasks involved in assessing brain health. In some embodiments, speech task screen 530 may also comprise button 545, which, upon interacting with by the participant and/or user, causes client device to record the sounds made by the participant and/or user. In some embodiments, button 545 may be animated, the animation may indicate the duration of recording, how much required time is left to record for a particular speech task and/or recording session, or it may indicate that recording as started.

FIG. 5C is a screen shot of a speech task screen 550, according to some embodiments. Speech task screen 550 is an example of a speech task and/or recording task that is currently being recorded, as indicated by button 555. In some embodiments, button 555 may be interacted with when the speech task and/or recording task is completed and/or when the participant and/or user wishes to stop and/or pause recording. Button 555 may also, in some embodiments, be animated, and the animation may indicate the duration of recording, how much required time is left to record for a particular speech task and/or recording session, or it may indicate that recording has started. Speech task screen 550 may also comprise recording timer 560, configured to indicate the progression and/or total recorded time of the speech task or recording task.

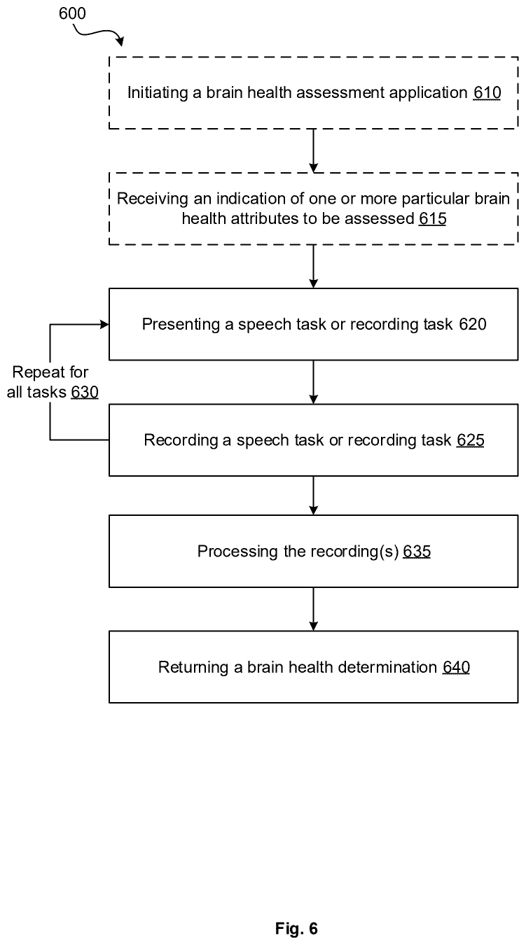

FIG. 6 is a process flow diagram of a method 600 of determining brain health performed by system 200, according to some embodiments. Optionally, at 610, a participant and/or user assisting the participant may initiate, start and/or otherwise access the assessment application 225 via mobile computing device 210. The assessment application 225 may be downloaded and installed on mobile computing device 210, via network 240. Assessment application 225 may be a web application, that is run via a web browser (not shown) installed on client device 210. Upon initiating assessment application 225, the participant and/or user may be presented with a login screen, wherein they may either log in with an existing account, or create a new account. In some embodiments, each participant may have their own account, or a medical institution and/or medical practitioner may have their own account, and each participant/patient may have a user profile associated with the account.

In some embodiments, at 615, the system 100 may receive an indication of one or more brain health attributes that are to be assessed by the system 100. In some embodiments, the participant and/or user may be presented with a data entry field and/or list of brain health attributes/conditions to be assessed. The selection of a particular brain health attribute may determine which speech tasks and/or recording tasks are presented to the participant and/or user, and/or which speech tasks and/or recording tasks are available for presentation to the participant and/or user. In some embodiments, the selection of a particular brain health attribute may determine one or more machine learning model(s) to be used for the processing of the recorded speech tasks and/or sounds. In some embodiments, the selection of a particular brain health attribute may be used to configure one or more data filtering processes, such as a sliding data selection filter. The data filtering process may select or filter out aspects of the recorded sounds and/or textual representation of the recorded sound to more accurately assess for the particular brain health attribute.

At 620, the participant and/or user is presented with a speech task and/or recording task. The speech task may comprise speaking a certain word, making a certain sound, holding a conversation and/or saying a word or words according to certain criteria (e.g. as many rhyming words as the participant can think of). The recording task may comprise recording the sounds made by a participant for an extended period of time.

At 625, the speech task or recording task is recorded by system 100. In some embodiments, the task is recorded by the client device 210. The client device may comprise device I/O 235, configured to record audio, such as an integrated microphone. In some embodiments, client device 210 may be a ‘kiosk’ comprising a computing device connected to one or more microphones, connected via an audio connection, such as USB, XLR and/or TRS. At 630, once a speech task or recording task is completed, the system 100 may present the next, or subsequent speech task or recording task to be completed by the participant and/or user.

At 635, the one or more recordings are processed, according to method 400, to generate a determination of brain health. In some embodiments, the recordings are communicated to audio analysis server 250 for processing via network 240. In some embodiments, client device 210 may be configured to process the recordings to determine brain health. In some embodiments, the audio recordings and/or the brain health determinations may be communicated to and stored on database 245.

At 640, a determination of the participant's brain health is returned. The determination may be a diagnosis of a certain brain health condition and/or neurological condition. The diagnosis may be a binary determination of whether a participant is likely to have a certain condition. In some embodiments, the determination may be a numerical value. The numerical value may be on a scale of condition severity, or likelihood of a participant having a certain condition. In some embodiments, the determination may comprise a plurality of numerical values, each numerical value associated with a particular aspect of brain health. The numerical values may represent a value on a scale, the scale being associated with a proficiency, deficiency, and/or progression of a brain health condition and/or neurological disorder.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

It will be appreciated by persons skilled in the art that any particular method and/or technology that is described in relation to or used in combination with any one or more individual element or elements in the above-described embodiments may equally be applied to any other applicable elements described above, without departing from the broad general scope of the present disclosures.

1. A computer implemented method comprising:
receiving an audio recording of sounds made by a participant;
determining, from the audio recording, a textual representation of at least one sound made by the participant;
providing the audio recording to an acoustic analysis model;
receiving from the acoustic analysis model, a speech quality dataset comprising one or more speech quality metric(s);
providing the textual representation to a text analysis model;
receiving from the text analysis model, a speech content dataset comprising one or more speech content metric(s); and
determining, from the speech quality dataset and the speech content dataset a speech score associated with the participant;
wherein the speech score is indicative of a brain health of the participant.

2. The computer-implemented method of claim 1, wherein the speech score comprises at least a naturalness score.

3. The computer-implemented method of claim 2, wherein the naturalness score includes at least one measure of: fundamental frequency variability, intensity variability, formant transitions, speech rate and rhythm, phonation measures, spectral measures, temporal measures, articulation, coarticulation, resonance and prosody.

4. The computer-implemented method of claim 1, wherein the speech score comprises at least an intelligibility score.

5. The computer-implemented method of claim 1, wherein the speech score comprises one or more: communication effectiveness score, dysarthria score, disease severity score, social communication score, and/or voice quality score.

6. The computer-implemented method of claim 1, wherein the speech quality dataset comprises one or more: timing metric, articulation metric, resonance metric, prosody metric and/or voice quality metric.

7. The computer-implemented method of claim 1, wherein the speech content dataset comprises at least a discourse complexity metric.

8. The computer-implemented method of claim 7, wherein the discourse complexity metric includes at least one of: lexical diversity, syntactic complexity, referential cohesion, thematic development, argumentative structure, implicit and explicit information comparison, interactivity, intertextuality, modality and modulation and pragmatic factors.

9. The computer-implemented method of claim 1, wherein the speech content dataset comprises one or more: semantic complexity metrics, idea density metrics, verbal fluency metrics, lexical diversity metrics, informational content metrics, discourse structure metrics, and/or grammatical complexity metrics.

10. The computer-implemented method of claim 1, further comprising:
comparing the speech score associated with the participant to one or more previous speech scores associated with the participant, to determine a change in the brain health of the participant; or
comparing the speech score associated with the participant, to a control speech score to determine the brain health of the participant.

11. (canceled)

12. The computer-implemented method of claim 10, wherein the control speech score is experimentally determined or statistically determined.

13. The computer-implemented method of claim 1, wherein the sounds made by a participant are spoken words and/or sounds; and
wherein the spoken words and/or sounds are in response to a speech task provided to the participant and/or the spoken words and/or sounds were recorded over a period of continuous observation of the participant without being prompted.

14. The computer-implemented method of claim 1, further comprising performing data quality assurance, wherein the data quality assurance comprises one or more of:
(a) determining if a voice is present:
(b) determining a recording duration;
(c) determining the recording duration is within a predetermined recording limit;
(d) remove aberrant noise;
(e) remove silence;
(f) determine the number of recorded speakers;
(g) separate speakers.

15. (canceled)

16. (canceled)

17. The computer-implemented method of claim 1, wherein the determination of the speech content score and the speech quality score occur in parallel.

18. The computer-implemented method of claim 1 further comprising:
receiving one or more narrowing parameters, wherein the one or more narrowing parameters comprises:
one or more brain health conditions;
one or more brain health attributes;
one or more brain health metrics;
one or more neurological disorders; and/or
participant medical history.

19. (canceled)

20. The computer-implemented method of claim 18 wherein:
responsive to receiving one or more narrowing parameters, adjusting the acoustic analysis model and/or the text analysis model such that the speech quality dataset and/or the speech content dataset are associated with the one or more narrowing parameters.

21. The computer implemented method of claim 1, wherein one or more of the textual representation, the speech quality dataset, the speech content dataset and the speech score are determined by one or more machine learning model(s).

22. (canceled)

23. (canceled)

24. The computer implemented method of claim 1, further comprising:
before the audio recording is provided to the acoustic analysis model, converting the audio recording into one or more of a series of filter-bank spectra and/or one or more sonic spectrogram.

25. (canceled)

26. (canceled)

27. (canceled)

28. (canceled)

29. A non-transitory machine-readable medium storing instructions which, when executed by one or more processors, cause a computing device to perform the method of claim 1.

30. A system for assessing brain health comprising:
a speech analysis module configured to determine a speech quality dataset from an audio recording;
a speech to text module configured to determine a textual representation of the audio recording;
a speech content module configured to determine a speech content dataset from the audio recording;
a brain health determination module configured to determine, using the speech quality dataset and the speech content dataset, a speech score;
wherein the speech score is indicative of the brain health of the one or more participants recorded on the audio recording:
wherein the system further comprises
a screen, configured to present instructions to the one or more participants; and
an audio recording device, configured to record the one or more participants.

31-37. (canceled)
